(19) United States
(12) Patent Application Publication
(10) Pub. No.: US 2024/0228926 A1
(43) Pub. Date: Jul. 11, 2024

(54) MICROWELL ARRAY FOR HIGH-THROUGHPUT SCREENING OF MICRO-TISSUE AND METHODS OF USING THE SAME

(71) Applicant: Organos, Inc., Berkeley, CA (US)

(72) Inventors: Kevin E. Healy, Moraga, CA (US); Nathaniel Huebsch, Clayton, MO (US); Julia Schaletzky, EI Cerrito, CA (US); Samuel Wall, Oslo (NO)

(73) Assignee: Organos, Inc., Berkeley, CA (US)

(21) Appl. No.: 18/570,952

(22) PCT Filed: Jun. 16, 2022
(86) PCT No.: PCT/US2022/033823

§ 371 Date: Dec. 15, 2023

Related U.S. Application Data
(60) Provisional application No. 63/211,645, filed on Jun. 17, 2021.

(51) Int. Cl. C12M 3/00; C12M 1/12; C12M 1/32; C12M 1/42

(52) U.S. Cl. CPC C12M 21/08; C12M 23/12; C12M 25/00; C12M 35/02

(57) ABSTRACT

A microwell array configured for high-throughput screening of micro-tissue and methods of using the same to prepare micro-tissue is disclosed. The microwell array includes a substrate having a plurality of wells, at least one longitudinal recess arranged in the bottom surface of each of the plurality of wells, and at least one micropillar arranged within the longitudinal recess at each end of the longitudinal recess. The microwell array advantageously can provide for high-throughput screening by enabling in vitro generation of three-dimensional micro-tissues that are accurate models of heart, skeletal muscle, neuronal, and other tissues in a device compatible with existing robotic liquid handlers to load cells into the devices, perform routine media changes, and add molecular probes and compounds when desired.

### TECHNICAL FIELD

This disclosure relates to a microwell array for high-throughput screening of micro-tissue and methods of using. The microwell array advantageously can provide for high-throughput screening by enabling in vitro generation of three-dimensional micro-tissues that are accurate models of heart, skeletal muscle, neuronal, liver, and other tissues in a device compatible with existing robotic liquid handlers to load cells into the devices, perform routine media changes, and add molecular probes and compounds when desired.

### BACKGROUND

Although heart failure is the leading cause of death in the U.S., therapeutic treatments remain suboptimal as the pharmaceutical industry remains incapable of generating more predictive human-relevant preclinical models of the human heart that are amenable to high-throughput screening (HTS) assays. Despite recent improvements in R&D productivity, large compound libraries remain virtually unexplored in a predictive cardiac tissue model, hindering rapid discovery of therapeutic approaches to treat heart disease. It is a strong belief that the ability to optimally identify efficacious drug candidates will come from HTS assays on healthy and diseased human heart tissues that generate data with high dynamic range and Z-factor.

To effectively use in vitro tissue models for the types of extensive tests needed to evaluate the safety and efficacy of drugs, the tissues should be three-dimensional, they should exhibit appropriate physio-chemical properties, they should have dimensions relevant to features of the tissues of interest, and the cells within the tissues should be differentiated and reproducibly express relevant biomarkers. In addition, the tissue models should be available in sufficient numbers for statistically relevant studies, and employ cell numbers and cell types that can reasonably be generated with a reproducible phenotype and purity.

U.S. Pat. No. 10,851,344 proposes methods and devices for preparing a tissue model of cardiac function that exhibits beating frequency, beating strength, electrical activity, and different channel activities of functional human cardiac tissue. However, contractility analysis on the tissue model requires removal of the tissue from the culture device and mounting on an external instrument, which is not conducive to HTS. Additionally, the disclosed systems are not compatible with existing HTS equipment, such as robotic liquid handling devices.

Accordingly, there is a need for systems enabling both efficient and robust preparation of in vitro tissue models and HTS of the same.

### SUMMARY

We provide a microwell array configured for high-throughput screening of micro-tissue comprising:
a substrate;
a plurality of wells in the substrate, each well having an opening, a sidewall surface and a bottom surface;
at least one longitudinal recess arranged in the bottom surface of each of the plurality of wells; and
at least one micropillar arranged within the longitudinal recess at each end of the longitudinal recess.

### BRIEF DESCRIPTION OF THE DRAWINGS

FIG. 1 shows an exemplary microarray device.

FIG. 2 shows a schematic of an exemplary well of the microarray device.

FIG. 3 shows a schematic of micro-tissue assembled in an exemplary longitudinal recess.

FIG. 4A shows a perspective schematic of an alternate exemplary well of the microarray device. FIG. 4B shows a cross-sectional schematic of an alternate exemplary well of the microarray device.

FIG. 5 shows an SEM of an exemplary arrangement of micropillars in an example with four dog-bone wells.

FIG. 6 shows an SEM of an exemplary arrangement of micropillars with an alternative example of dog-bone wells.

FIG. 7A shows heart micromuscles indicated by fluorescent dye formed on four dog-bone wells.

FIG. 7B shows the contraction of the heart micromuscle as detected by measuring changes in voltage, calcium and motion over time.

FIG. 8 shows an exemplary field of view of an exemplary microwell for high frame-rate imaging analysis.

FIG. 9A shows a cross-sectional schematic of a tapered micropillar to illustrate bending beam theory of a uniform material stress applied to the tapered pillar and the resulting deflection this produces at the tip of a tapered pillar.

FIG. 9B shows a graph of calculated afterload (maximum contraction stress) on tapered micropillars as a function of pillar diameter for microtissues grown in COC molded wells.

FIG. 10 shows two alternate exemplary arrangements of microwells.

### DETAILED DESCRIPTION

This disclosure provides devices and methods for HTS of in vitro-generated three-dimensional tissues that are accurate models of heart, skeletal muscle, and other contractile tissues. Such models are useful for tests involving pharmacological efficacy, safety and toxicity studies. The models can include mixtures of cells that would commonly be present in an organ or tissue of interest that self-assemble into three-dimensional cellular structures. Advantageously, such accurate organ and tissue models can be manufactured by culturing cells in a device compatible with robotic liquid handling and without adding exogenous matrix or biomaterials.

The devices provided herein are useful for forcing cells to become aligned and to self-assemble into three-dimensional tissues on a scale amenable to HTS. The devices generally include a microwell array having a plurality of wells (also referred to as microwells), at least one longitudinal recess arranged in the bottom surface of each of the plurality of wells and at least one micropillar arranged at opposing ends of the longitudinal recess.

Together, the microscale geometry of the longitudinal recess and positioning of the micropillars facilitates cell alignment and self-assembly into micro-tissues by the longitudinal recess being deep enough to hold sufficient cells and narrow enough to force cellular alignment and three-dimensional self-assembly and by the micropillars anchoring opposing ends of the micro-tissues. Thus, cells uniaxially align in the longitudinal recess and form a tissue with a local gradient of mechanical stress, as the cells are guided by geometrical cues from the recess. Additionally, the tissue arranges itself on the micropillars in a manner suitable for testing in situ.

Although each of the longitudinal recesses is small enough to be seeded with only about 1,000-10,000 cells, the tissues that self-assemble within the longitudinal recesses accurately and realistically model the properties of in vivo tissues. For example, cardiac micro-tissues formed using the devices and methods described herein express biomarkers of mature cardiac tissues, exhibit highly synchronous contractility, and respond to drugs in the same manner as heart tissues (e.g., with synchronous chronotropic and/or inotropic responses).

The low volume of the longitudinal recesses and the small sizes of the micro-tissues are advantageous because the types of cells needed for evaluation eliminates the need to grow up large numbers of cells, and a multitude of micro-tissues can simultaneously be generated and tested. Thus, the devices allow high throughout testing with statistically significant numbers of tissues. Abundant control micro-tissues can also be generated and tested as desired.

Alignment of cells and contractile force within the tissues occurs because of the geometric constraints of the longitudinal recesses. When seeding cells into the device one would generally expect that the cells within the microwells would initially exert the same amount of traction force per cell, and exert stress in a random direction. However, when all the force vectors are added together for the traction exerted by all cells due to the device geometry, the magnitude of the net force along the longitudinal axis of the longitudinal recess is much greater than the net force along the transverse axis.

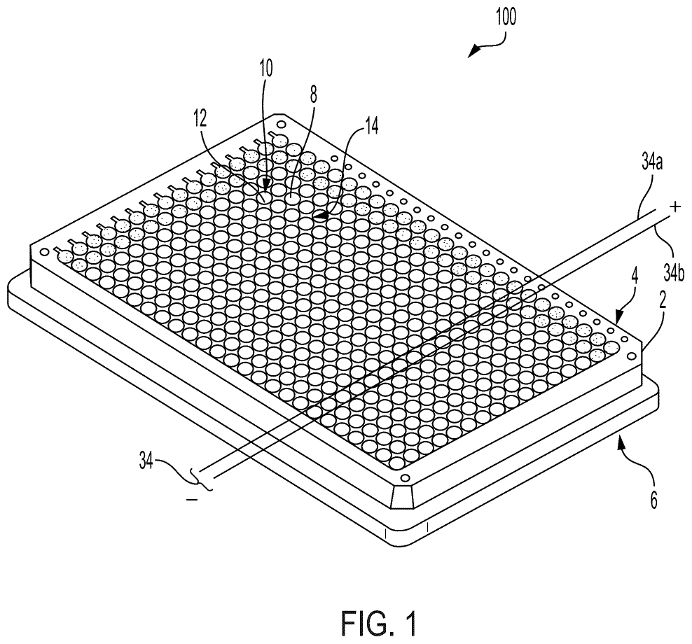

Referring now to FIG. 1, the microwell array 100 can comprise a substrate 2 with upper surface 4 and lower surface 6. The substrate 2 further can comprise a plurality of microwells 8 extending between the upper surface 4 and lower surface 6 of the substrate 2. Each microwell 8 in the microwell array 100 can comprise an opening 10, a bottom surface 12 and at least one side wall 14 connecting to the bottom surface 12 and extending between the opening 10 and bottom surface 12.

The number of microwells in the microwell array is not particularly limited. Advantageously, the microwell array can be configured with a total of 6, 24, 96, 384, or 1536 microwells, preferably arranged in a 2:3 rectangular matrix. In certain examples, the microwell array comprises from 6 to 1536 microwells (e.g., from 6 to 384 microwells, or from 6 to 200 microwells). In certain examples, the microwell array comprises 6, 24, 96, 384, or 1536 microwells.

In some examples, the microwell array comprises an array of microwells which together and individually have one or more dimensions, including well diameter, well spacing, well depth, well placement, plate dimensions, plate rigidity, and combinations thereof, equivalent to the standard dimensions for microwell plates published by the American National Standards Institute (ANSI) on behalf of the Society for Biomolecular Sciences (SBS). See, for example, Journal of Biomolecular Screening, Vol. 1, Number 4, 1996, pp. 163-168, which is incorporated herein by reference for its description of the standard dimensions of multi-well plates. In this way, the array of microwells can be rendered compatible with existing technologies for HTS, including multi-channel micropipettes, robotic liquid handlers, automated plate readers, and the like.

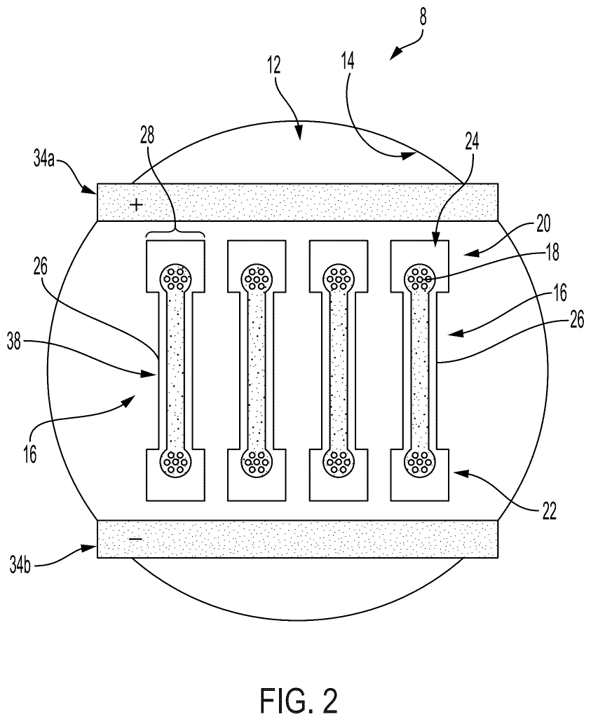

As shown in FIG. 2, each microwell 8 is provided with at least one longitudinal recess 16 arranged in the bottom surface 12 of each of the plurality of wells 8 and at least one micropillar 18 arranged at each of the opposing ends (20 and 22) of the longitudinal recess 16.

As a longitudinal recess 16 is a recess within the well 8, the longitudinal recess 16 extends in a depth direction between the bottom surface 12 of the well 8 and a bottom surface 24 of the longitudinal recess 16. Each longitudinal recess 16 can comprise a recess opening 35, a recess bottom surface 36 and at least one recess side wall 37 connecting to the bottom surface 12 and extending between the opening 35 and bottom surface 36.

Suitably, a depth of a longitudinal recess 16 relative to the bottom surface 12 of the well may be about 500 μm or less, or about 400 μm or less, or about 300 μm or less, or about 200 μm or less or about 150 μm or less.

Typically, at least a portion of a longitudinal recess 16 may have a configuration as a groove portion 26. Preferably, the groove portion 26 of the longitudinal recesses 16 is configured as a linear (e.g., straight, angled or curvilinear) indentation.

In some optional examples, one or both of the opposing ends (20 and 22) of a longitudinal recess 16 may be further provided with an expanded depression 28 having a width greater than a width of the groove portion 26.

In examples with two expanded depressions 28, the groove portion 26 extends between and connects the expanded depressions 28 to form a “dog-bone” shaped longitudinal recess 16. Here, the term width refers a dimension perpendicular to a longitudinal axis of the longitudinal recess 16. A longitudinal recess 16 including two opposing expanded depressions 28 may have a “dog-bone” geometry, where the expanded depressions 28 correspond to “knobs” at the end of the dog-bones and can be connected by the groove portion 26 corresponding to a “shaft” of the dog-bone. In view of this general shape, a longitudinal recess 16 may also be referred to herein as a “dog-bone well.”

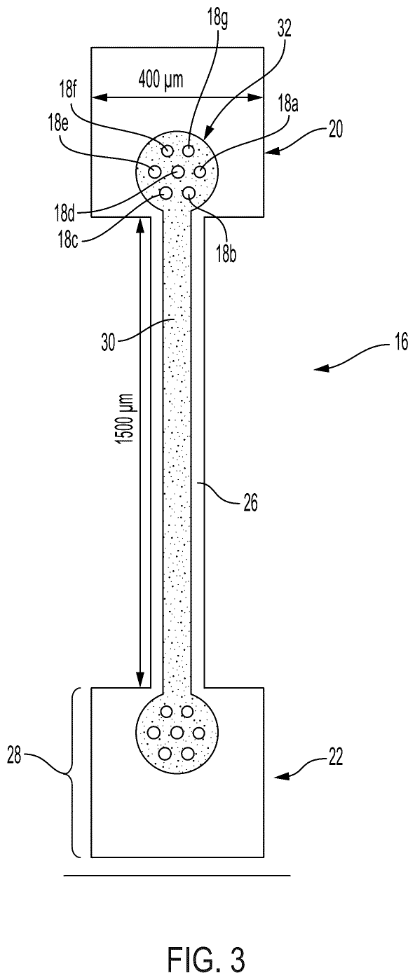

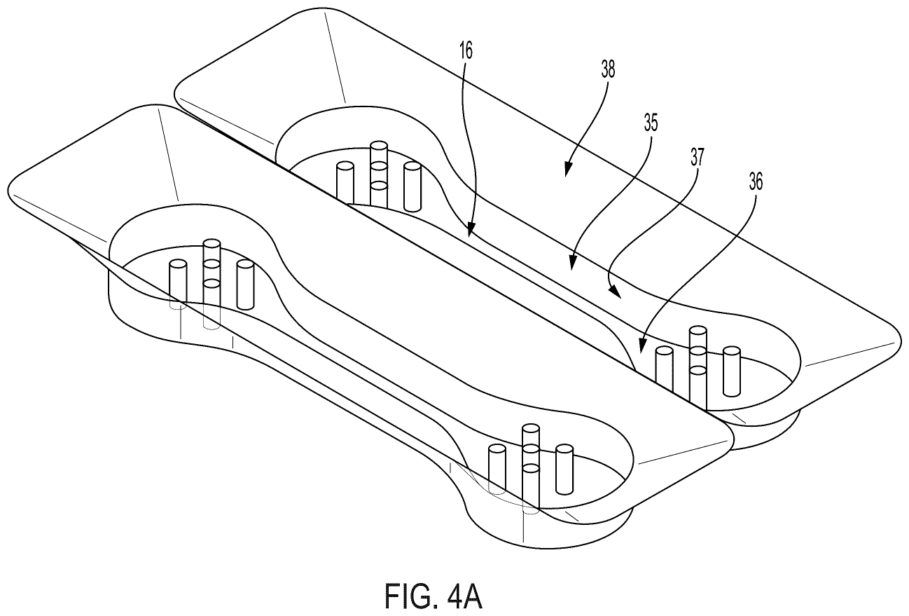

In examples with or without expanded depressions 28, the microarray 100 includes at least one micropillar 18. As shown in FIG. 3, the micropillars 18 are structures projecting substantially perpendicularly from the bottom surface 24 of the longitudinal recess 16. The micropillars 18 are arranged within the longitudinal recess 16 at each opposing end (20 and 22) of the longitudinal recess 16. As shown in FIG. 3, each end (20 and 22) of the longitudinal recess 16 may comprise seven micropillars 18a-18g arranged in a hexagonal pattern with a central pillar. FIGS. 4A and 4B depict perspective views of a similar hexagonal arrangement. The microarray 100 may include at least two, three, four, five, six, seven, eight or nine micropillars at each opposing end (20 and 22) of the longitudinal recess 16.

Suitable cross-sectional geometries of the micropillars include circular, oval, polygonal and like shapes. The upper surface of a micropillar facing the opening of the well 8 may be flat, concave, convex, or other geometries.

The micropillars advantageously provide support to anchor a micro-tissue 30 in the longitudinal recess 16 after the micro-tissue self-assembles in the area 32 surrounding the micropillars. Even more advantageously, the micropillars provide resistance to the micro-tissue contraction and can be used to measure contractility and force production of the micro-tissue in situ. In other words, the micro-tissues can be both formed and subjected to tissue analysis in the microarray. The ability to perform cell culture and micro-tissue analysis in the same device and without transferring the tissue to a second device dramatically increases efficiency relative to existing tissue modelling techniques and is highly advantageous to HTS.

The contractility of the micro-tissue can be measured in situ by evaluating the force or contraction applied to the micropillars. When an axially oriented micro-tissue mounted within a longitudinal recess 16 is caused to contract, the micro-tissue applies a force to the micropillars. The force of contraction applied to a micropillar configured with a suitable elastic modulus causes the micropillar to physically deflect or bend. The force of contraction may be calculated by optically capturing the deflection of the pillars (e.g., by using an ImageXpress® Micro Confocal High-Content Imaging System) and then applying Euler-Bernouli beam theory. Measurement of force using mechanosensing pillars is described in U.S. Pat. No. 10,233,415, which is incorporated herein by reference in its entirety.

As the material selection can influence the elastic modulus of the micropillar and dimensions of the micropillars can influence the stiffness, those properties may be selected to achieve the desired resistance to the heart muscle contraction. For example, for a cyclic olefin copolymer, a suitable diameter of the micropillars may be 5 μm to 100 μm, more preferably 20 μm to 80 μm, more preferably 30 μm to 70 μm. The diameters of the micropillars of different longitudinal recess 16 may be uniform or varied. As shown in FIG. 4, longitudinal recesses 16 arranged in single well 8 may be provided with micropillars 18 of successively smaller size. Such an arrangement with micropillars 18 of successively smaller size advantageously allows for comparative evaluation of the force of contraction of micro-tissue on micropillars with difference stress characteristics.

While the term “diameter” is used, it should be understood that the geometry of the micropillars is not limited to a cylinder and that the term “diameter” may describe a length or width of micropillars configured with a rectangular, triangular or other cross-sectional shape.

Furthermore, micropillars 18 may have sides that are straight vertical or tapered. In a tapered micropillar, a diameter (d_{A}) of the micropillar 18 at a tip of the micropillar 18 may be less than a diameter (d_{B}) of the micropillar 18 at a base of the micropillar 18 proximal to the bottom surface 24 of longitudinal recess 16. Preferably, an angle of tapering of a tapered micropillar 18 may be between 1° and 10°, or preferably between 2° and 4°, relative to a plane orthogonal to the bottom surface 24 of longitudinal recess 16. In such examples of tapered micropillars 18, the value of the diameter dimension refers to a diameter d_{B} at a base of the micropillar proximal to the bottom surface 24 of longitudinal recess 16.

The height of the micropillars 18 is not particularly limited, although a height less than or equal to the depth of the longitudinal recess 16 is convenient for manufacturing. Thus, the height of the micropillars 18 (L) extending from the bottom surface 24 of longitudinal recess 16 may be about 500 μm or less, or about 400 μm or less, or about 300 μm or less, or about 200 μm or less or about 150 μm or less. The height of the micropillars 18 extending from the bottom surface 24 of longitudinal recess 16 may be about 10 μm or more, 20 μm or more, 30 μm or more, 40 μm or more, 50 μm or more, 60 μm or more, 70 μm or more, 80 μm or more, 90 μm or more or 150 μm or more, 150 μm or more or 200 μm or more.

Preferably, each of the opposing ends (20 and 22) of the longitudinal recess 16 is provided with a plurality of micropillars 18. Generally, the positioning of the micropillars 18 may be confined to the end regions 20 and 22 of the longitudinal recess 16, such as in expanded depressions 28 if present, such that a middle portion 40 of the longitudinal recess 16 is free of pillars. Restricting the positioning of the micropillars 18 to the end regions 20 and 22 of the longitudinal recess 16 advantageously allows for anchoring of the ends of micro-tissue without interfering with contractile movements of the micro-tissue.

The number of micropillars is not particularly limited and can be 2, 3, 4, 5, 6, 7 or more. When a plurality of micropillars are included, they may be arranged in each depression in a circular, polygonal or hexagonal pattern. A preferred arrangement is a hexagonal pattern having a central pillar.

To facilitate measurement of contraction, the microarray can include a set of electrodes 34 in electrical contact with one or more wells 8. The set of electrodes 34 includes a positive electrode 34A and a negative electrode 34B. Each of the electrodes 34A/34B, are electrically isolated from one another and, when supplied an electric current, can create an electrical field within the well that is useful for pacing contraction of micro-tissues therein.

As shown in FIG. 2, the positive electrode 34A and a negative electrode 34B may each be a strip of electrode material overlaying the upper surface 4 of the substrate 1 and spanning the opening 10 of a well 8. With this configuration, the well 8 can be filled with a liquid medium such that the liquid medium in the well 8 comes into contact with the electrodes 34. Contact between the electrodes 34 and a liquid medium filling the well 8 conducts the electric field to the well 8 and micro-tissues therein.

As suitable configuration of the electrodes 34 may include strips of electrode material extending across a plurality of wells 8, such as adjacent wells 8 in a row of wells in the microarray 100. In such configuration, each row may be respectively provided with a set of electrodes 34.

The electrodes 34 may be connected to the substrate 1 by any suitable means that maintains the electrodes 34 electrically isolated from one another and that does not interfere with their ability to apply and electrical field in the wells 8.

The electrodes 34 may be prepared from any suitable conductive material, including but not limited to indium tin oxide, gold, platinum black, platinum, graphene, or conductive polymers such as poly(3,4-ethylenedioxythiophene) polystyrene sulfonate (PEDOT:PSS), and may have any suitable geometry and/or dimensions. In some examples, the electrodes 34 may be comprised of evaporated gold.

To advantageously increase capacity for HTS, one or more wells 8 may be provided with multiple longitudinal recesses 16. The number of longitudinal recesses 16 per well 8, while not particularly limited, may be constrained by the size limitations of the well and tolerance of microforming techniques. For example, in a microwell array having dimensions equivalent to that of a standard 384-microwell plate, each well may accommodate four longitudinal recesses, allowing for an array containing 1,536 tissue models.

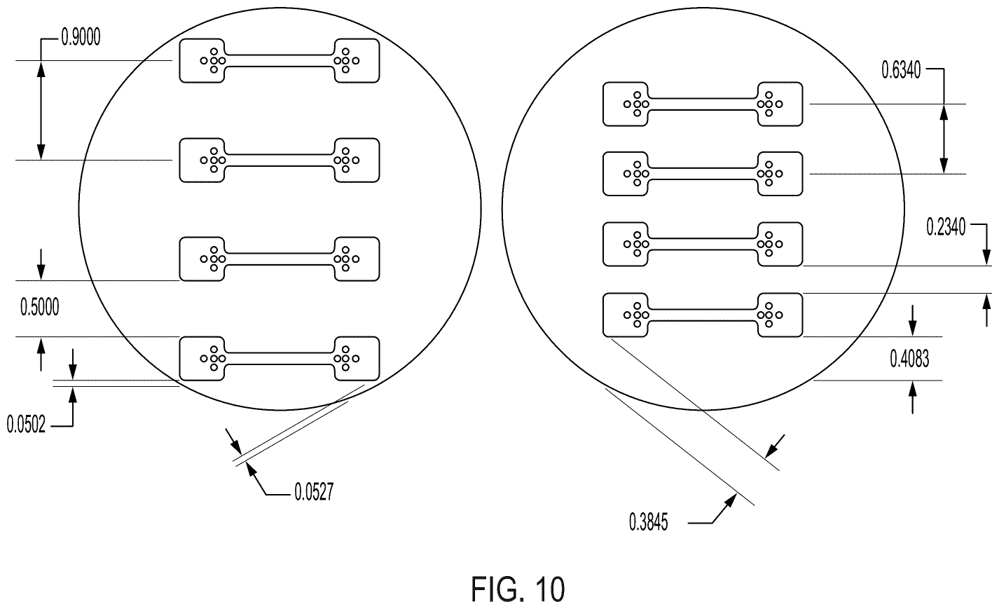

In examples of wells containing a plurality of longitudinal recesses, the multiple longitudinal recesses 16 may be arranged parallel to each other and each longitudinal recess is preferably discontinuous from adjacent longitudinal recesses. In other words, adjacent longitudinal recesses may be spatially separated from one another by a portion of material forming the bottom surface of the well. A single well may contain at least two, three, four, five, six, seven, eight or nine longitudinal recesses 16. FIG. 10 shows two alternate exemplary arrangements of microwells with four longitudinal recesses in each well (dimensions shown in mm).

In examples of longitudinal recesses 16 having expanded depressions 28, the larger width of the expanded depressions 28 relative to the groove portion 26 of the longitudinal recess 16 may facilitate anchoring of the micro-tissues so that, during contraction of the tissues along the longitudinal axis of the longitudinal recesses 16, the tissues are less likely to become detached or torn away from the micropillars 18. For example, the width of the groove portion 26 is typically about 1:3 to about 1:10, or about 1:3 to about 1:7, or about 1:3 to about 1:5, or at least about 1:4 of the width of the expanded depressions. The groove portions can, for example, be about 10 μm to about 250 μm wide, or about 20 μm to about 225 μm wide, or about 30 μm to about 200 μm wide, or about 40 μm to about 175 μm wide, or about 50 μm to about 150 μm wide, or about 60 μm to about 135 μm wide, or about 70 μm to about 130 μm wide, or about 75 μm to about 125 μm wide, or about 100 μm wide. The length of the groove portion can vary from about 100 μm to about 2000 μm, or from about 200 μm to about 1500 μm, or from about 300 μm to about 1000 μm, or from about 400 μm to about 700 μm.

The expanded depressions 28 can be about as long as they are wide. However, some variation from a 1:1 ratio of expanded depression 28 width to length is acceptable, and in some cases such variation is desirable. For example, the length compared to the width of the expanded depressions 28 can be about 1:1.5, or about 1:1.25, or about 1:1, or about 1.15:1, or about 1.25:1, where the length is measured along the longitudinal axis of the longitudinal recess 16, and the width is measured perpendicular to the longitudinal axis.

The expanded depressions 28 of the longitudinal recess 16 can have a substrate surface area of about 50 μm² to about 500,000 μm², or of about 100 μm² to about 250,000 μm². The volume of the expanded depressions 28 can vary. For example, the volume of the expanded depressions 28 can be about 0.05 μL to about 2 μL, or about 0.1 μL to about 1.0 μL, or about 0.1 μL to about 0.5 μL.

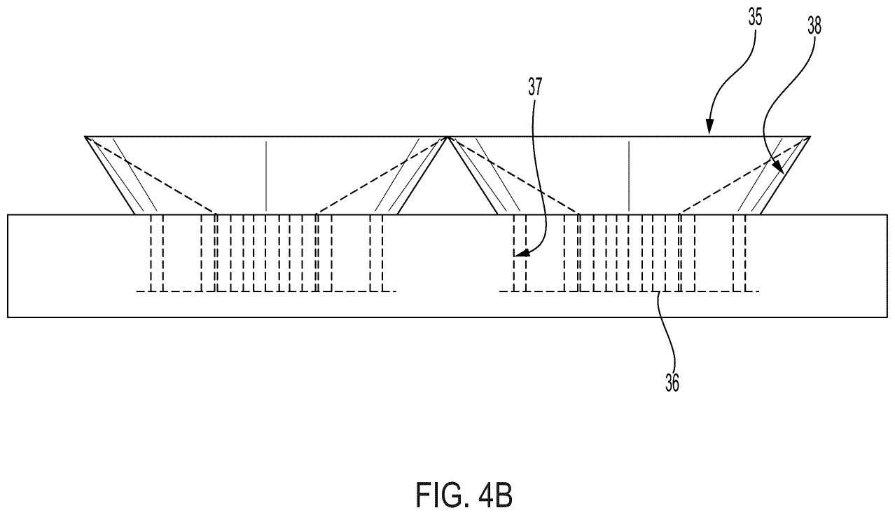

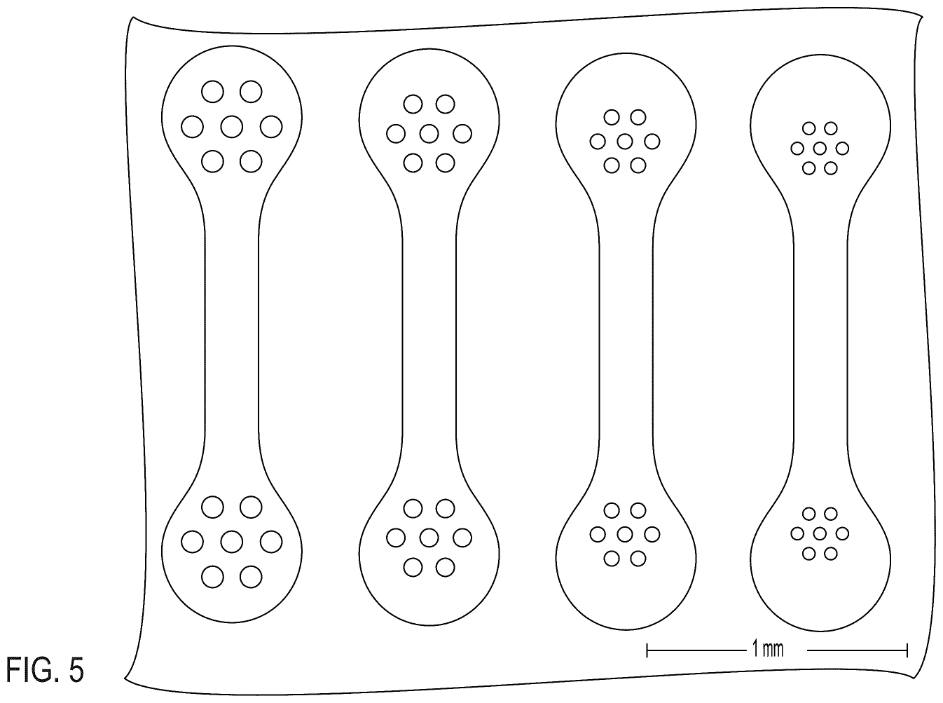

In addition, expanded depressions 28 may have angled corners or rounded sides. Hence, expanded depressions 28 may be square, rectangular, triangular, Y-shaped, T-shaped, angular, circular or oval-shaped. FIG. 5 shows a dog-bone well manufactured with exemplary rounded expanded depressions 28 and FIG. 6 shows a dog-bone well manufactured with exemplary rectangular expanded depressions 28.

In some examples, the bottom surface 12 of the well 8 may define a substantially planar, horizontal surface within which the one or more longitudinal recesses 16 are formed. In other examples, as best seen in FIGS. 4A and 4B, a portion of the bottom surface 12 of the well 8 connecting to the recess side wall 37 may be provided with a sloped portion 38 having an angle of incline such that a depth of the bottom surface 12 of the well 8 in the sloped portion 38 increases with closer proximity to the longitudinal recess 16 and is greatest where the bottom surface 12 connects with the recess side wall 37 of the longitudinal recess 16. As shown in FIG. 4A, a sloped portion 38 may preferably surround each longitudinal recess 16. FIG. 4A shows adjacent longitudinal recesses 16 that are surrounded by respective sloped portions that join together to form an apex, as shown in FIG. 4B. However, it is also possible for the sloped portions 38 to be separated, such as by a horizontal portion of the bottom surface 12 of the well. Advantageously, due to gravity, the sloped portion 38 helps direct cells provided into the well 8 into the longitudinal recesses 16, thereby increasing the number of cells that settle within the longitudinal recess 16.

The substrate 1 can have a cell adhesion coating to facilitate cellular adhesion to the substrate. Such a cell adhesion coating can include adhesion proteins such as fibronectin, vitronectin, E-selectin, gelatin, laminins, collagens, collagen type I or IV, or matrigel. The cell adhesion coating can also include hydrogel-forming polymers such as fibrinogen/fibrin, bisacrylamide, GelMA, or combinations thereof. In addition, the cell adhesion coating can include RGD peptides, PHSRN peptides, and DGEA peptides, FHRRIKA peptides and combinations thereof. Known methods of peptide discovery, such as bacterial display or the techniques disclosed in U.S. Pat. No. 8,501,905 (incorporated by reference herein), can be used to uncover unique peptides for cell adhesion. Preferably, the cell adhesion coating may be restricted to the area 32 surrounding the micropillars 18 so as not to interfere with movement or contraction of assembled micro-tissue.

Additionally or alternatively, the substrate 1 can be coated with a blocking agent or anti-adhesion coating to inhibit cell adhesion to the substrate. Suitably, a coating to reduce or prevent cell adhesion can be restricted to one or more of the groove portion 26 and/or middle portion 38 of the longitudinal recess 16, sidewalls of the microwell or dog-bone well or sloped portion above the dog-bone wells. Such a coating facilitates free movement of the micro-tissue during contraction.

A wide array of strategies may be used to modify the surface chemistry of the device's polymer. Suitable coatings for application by adsorption include proteins like bovine serum albumin, or natural and synthetic polymers such as alginate, polyethylene oxide, poly-N-isopropylacrylamide, block copolymers of poly(ethylene glycol) and poly(propylene glycol) (i.e. Pluronics), multi-arm poly(ethylene glycol), poly(styrene-co-3-sulfopropyl methacrylate) copolymers (Manfredini, N., et al. Industrial & Engineering Chemistry Research 58, 22290-22298 (2019)), or combinations thereof, or self-assembled layers (Ramasubramanian, A., et al. ACS Biomater Sci Eng 7, 1344-1360 (2021)). Alternatively, polymer systems that block protein adsorption and cell adhesion are known that can be chemically grafted to the device's polymer (e.g., COC). Examples include poly(ethylene glycol) (PEG)-based non-fouling polymer films (Ma, Z., et al. Nature Communications 6, 7413 (2015); Bearinger, J. P., et al. Langmuir 13, 5175-5183 (1997); and Jeon, H., et al. Journal Of The American Chemical Society 133, 6138-6141 (2011)), and perfluorodecanethiol grafting to the norbornene groups of the COC via thiol-ene click reactions. (Dichiarante, V., et al. ACS Sustainable Chemistry & Engineering 6, 9734-9743 (2018); Munoz, Z., et al. Biomater Sci 2, 1063-1072 (2014); and Lin, C. C., et al. J Appl Polym Sci 132 (2015)). Physical methods to prevent cell adhesion also exist, such as textured interfaces. (Jeon, H., et al. Nat Mater 14, 918-923 (2015)). Each of these methods, or some in combination chemistry plus physical methods can be optimized to minimize cell adhesion to devices base materials, allowing for optimal formation of independent (i.e., without syncytium between wells) heart micromuscle within a well of a well plate.

The blocking coating can be a polymeric coating, a protein coating, or a detergent. Examples of suitable blocking coatings include Pluronics, polyethylene oxide, alginate, poly-N-isopropylacrylamide, bovine serum albumin, or combinations thereof. The coatings can also include hydrogels such as bisacrylamide, alginate, agarose, polyethylene glycol diacrylate, or any combination thereof. Coatings may be applied by physio-absorption or covalent binding. The blocking agent may preferably be applied to the groove portion 26 and/or middle portion 40 of the longitudinal recess 16, although other surfaces of the substrate 1 may be coated as well.

The substrate 1 can include materials such as cyclic olefin copolymer (COC), polydimethylsiloxane (PDMS), surface functionalized PDMS, polyimide, polyurethane, SU8, thermoplastics, poly(methylmethacrylate) (PMMA), polycarbonate (PC), polystyrene (PS), polyethylene terephthalate (PET), polycaprolactone (PCL), poly(vinyl chloride) (PVC), glass, quartz, silicon, hydrogel forming polymers (e.g. hyaluronic acid, polyacrylamide, polyethylene glycol, alginate, agarose), protein-based gels (e.g., gelatin, collagen, and/or fibrin) or any combination thereof. Cyclic olefin copolymer (COC), such as TOPAS® Elastomer-E140, is preferred.

The groove and micropillars may be provided in the bottom surface of each of the plurality of wells by any suitable microforming technique, including micromachining, laser engraving, or micromolding.

Briefly, a suitable manufacturing process can be described as an injection molding process that creates micro-to nano-sized patterns. A master may be manufactured by laser lithography, copied into a Nickel—stamp by electroforming, and replicated in a disc format by a variothermal injection compression process. The process is similar to the production of an optical disc (CD, DVD or BD). The micro- to nano-sized patterns may be milled out of the disc substrate in a microscope slide format and laser-welded to a 96 or 384 well standard microplate frame, thus forming the bottom of the microplate array.

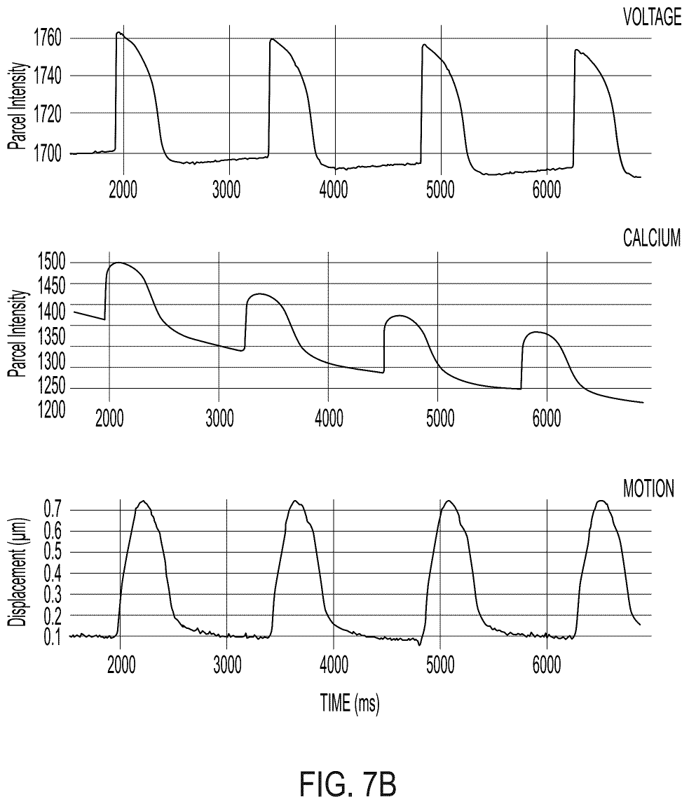

Aspects of the disclosure include systems that find use in conjunction with the microarray devices described herein. In some embodiments, a system includes a computational motion capturing component that is configured to image a plurality of cells that are cultured within a subject microarray device. In some embodiments, the computational motion capturing component is configured to detect a magnitude, velocity and/or direction of a motion made by a cell (e.g., a contraction of a cardiomyocyte or a region of the micro-tissue) with high spatial and temporal resolution. In some embodiments, the computational motion capturing component includes a camera that can capture images of cells that are cultured within one or more “dog-bone” wells of the device, and includes an algorithm that is adapted to determine a motion vector for a defined area of a captured image over a specified time interval. A high-speed camera may be used to image the microwell array wherein a field of view includes an end of a longitudinal recess, and more preferably multiple longitudinal recesses within one well. Additionally, for high frame-rate, the field of view may capture only one of the two expanded depressions at the ends of the longitudinal recesses and optionally omit imaging of the opposing end and connecting groove portion as shown in FIG. 8. In such examples, the field of view may have a length of 2 to 3 mm and height less than 500 μm.

In some embodiments, the algorithm can be used to determine a motion vector for a defined area of cells, or for an individual cell. Using the subject computational motion capturing component, the spatial distribution of the time-averaged movement velocity of a plurality of cells can be measured. In some embodiments, the computational motion capturing component can be used to determine absolute movement, as well as movement in a single coordinate direction, e.g., in the x-direction and/or in the y-direction. In some embodiments, the computational motion capturing component can be used to determine movement data as a function of time, as well as movement in a single coordinate direction, e.g., in the x-direction and/or in the y-direction, as a function of time. In some embodiments, the computational motion capturing system can be used to measure parameters such as mean contraction velocity, contraction angle, or mean contraction velocity in the x-direction and/or y-direction.

In some embodiments, the computational motion capturing component is configured to measure the displacement of one of more micropillars in the device. The displacement of a micropillar in the x- and/or y-direction can be used to determine the forces that are exerted on the micropillar by the cells that are cultured in the device, and can thus be used to conduct a mechanocardiogram analysis to determine, e.g., a beat rhythm for a plurality of cardiomyocytes that are cultured in the device. Image capture software can be used to determine the position of a micropillar as a function of time. The deflection or displacement of the micropillar is then determined by comparing the relative positions of the pixels in a plurality of images of the micropillar that are captured over a specified time period (e.g., a specified acquisition rate). In some embodiments, a plurality of images is collected at a rate of 100 frames per second for a total of 30 seconds.

Cells that can be cultured within microwells include partially and fully differentiated cells. Stem cells can also be cultured in the microwells, however, in general, partially and fully differentiated cells are desired for generation of micro-tissues that are accurate models of in vivo organs and tissues systems. Examples of cells that can be cultured to generate three-dimensional tissues include, but are not limited to, adipocytes, cardiomyocytes, fibroblasts, endodermal cells, epithelial cells, keratinocytes, myocytes, neurons, osteoblasts, pancreatic islet cells, retinal cells, stromal cells, macrophages, endothelial cells, pericytes, hepatocytes, stromal cells, and the like.

The cells that are cultured depend in part on the tissue type, or nature of the disorder or condition, to be tested. In general, at least some of the cells to be cultured naturally align, elongate, and/or contract in vivo.

The cells selected for culture in the microarray can be all of one cell type or be a mixture of cell types. For example, to optimally mimic an organ system, a mixture of the types of cells that found in the organ system can be employed. For example, in many cases heart disease is not caused by defects or injuries in cardiomyocytes themselves, but in fibroblasts, endothelial cells, neurons, or other cells that support the structure and function of the organ. The micro-tissues can be formed with defined mixtures cells, and mixing experiments can be performed where certain cell types are genetically labeled (e.g. to track calcium flux, sarcomere structure, or genetic modification), or where certain cell types have a defect that is associated with a disease or conditions via various mechanisms.

For example, if a three-dimensional micro-tissue model of the heart is desired, a mixture of cardiomyocytes, myoblasts, epithelial cells, endothelial cells, neuronal cells, fibroblasts, macrophages, stromal cells, pericytes, multipotent cardiomyocyte progenitors, or any combinations thereof can be employed. In another example, if a three-dimensional model of skeletal muscle is desired, a mixture of muscle tissue cells such as skeletal muscle stem cells, myoblasts, myosatellite cells, epithelial cells, myoepithelial cells, fibroblasts, connective cells, myoblasts, multipotent muscle progenitors, fibro-adipogenic progenitors, or any combinations thereof can be employed. In a further example, if a three-dimensional model of neuronal tissues is desired, a mixture of neurons, neuronal progenitor cells, glial cells, actrocytes, basket cells, beta cells, medium spiny neuron cells, pukinje cells, renshaw cells, unipolar brush cells, granular cells, anterior horn cells, spindle cells, and combinations thereof can be employed. Suitable techniques for growing a three-dimensional micro-tissue in a microenvironment are disclosed in U.S. Pat. No. 10,851,344, which is incorporated herein by reference in its entirety.

Such cell types can be obtained from a variety of sources. For example, the cells can be obtained from public cell depositories (e.g., Coriell Institute; the Allen Institute for Cell Science; the American Type Culture Collection, ATCC), from patients, from biopsies, via differentiation or conversion of other cell types, and any combination thereof. In some cases, the cells are obtained by differentiation from stem cells, or by conversion of one cell type for another.

To mimic various organ systems or disease state, the cells can be differentiated from stem cells of different genetic backgrounds, for example, by inducing formation of stem cells from somatic cells of patients with particular diseases or conditions.

Cells seeded within microwells can include at least some progenitor cells that mature as they grow, align, and self-assemble within the microwells.

For example, the stem cells can be induced pluripotent stem cells (iPSCs) or stem cells obtained from any convenient source. The stem cells can be at least partially differentiated or converted into the lineage of a desired organ or tissue type.

Examples of stem cells that can be employed include hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, and the like. Mammalian or human cells are preferred.

The devices described herein are useful for generating micro-tissues that realistically model in vivo organ and/or tissue systems. Methods of making such micro-tissues involve seeding selected mammalian cells into one or more microwells of a device described herein, and culturing the seeded cells within the microwells to thereby induce alignment and self-assembly of the mammalian cells into one or more micro-tissues.

The mammalian cells can be seeded in longitudinal recesses. Mammalian cells can be seeded in multiples recesses and/or multiple wells to permit generation of multiple micro-tissues useful, for example, for statistically relevant studies for testing micro-tissue models of selected organ or tissue types.

The microwell scale longitudinal recesses are typically seeded with fewer cells than are currently employed for toxicity, therapeutic agent identification, and drug testing. For example, some researchers have generated tissues from about 250,000 to about 1 million cells. However, the micro-tissues described herein can be generated from about 1000 to about 9,500 cells, or about 1500 to about 9000 cells, or about 2000 to about 7500 cells, or about 2500 to about 7000 cells per microwell.

The seeded cells are cultured for about 2 hours to about 14 days, or for about 1 day to about 10 days, or for about 2 days to 7 days, or for about 2 days to about 6 days, or for about 2 days to 5 days.

Culture media that can be employed include DMEM™, DMEM/F12, or Knock-Out (KO) DMEM available from Gibco (supplied e.g. by Gibco Invitrogen, Sigma, BD, Lonza) that contain low concentration of human or animal serum, or no serum, and bFGF, VEGF, ascorbic acid, heparin, and/or hydrocortisone as supplements. Another type of medium that can be mTESR-1 human pluripotent stem cell culture medium (STEMCELL Technologies), StemPro34 (Invitrogen), or EGM-2 BulletKit™ (Lonza). Non-limiting examples of optional factors than may be further included are insulin, IGF-I, hEGF, transferrin and/or hormones such as triiodothyronine.

For example, human ESCs and iPSCs can be cultured on Matrigel (BD Biosciences) coated plates with mTESR-1 human pluripotent stem cell culture medium (STEMCELL Technologies) to 80% confluence. Such cells can be dissociated with Accutase (Sigma) to small clumps containing 10-20 cells and resuspended in 2 ml basic media (StemPro34, Invitrogen, containing 2 mM glutamine, Invitrogen, 0.4 mM monothioglycerol, Sigma, 50 μg/ml ascorbic acid, Sigma, and 0.5 ng/ml BMP4, R&D Systems) to form embryoid bodies (EBs). Other factors such as BMP4 (10 ng/ml), human bFGF (5 ng/ml), and Activin A (3 ng/ml) can be added a day or a few days later to the basic media for cardiac specification. The media for embryoid bodies so formed can be replaced or refreshed with basic media containing human DKK1 (50 ng/ml) and human VEGF (10 ng/ml), followed by basic media containing human bFGF (5 ng/ml) and human VEGF (10 ng/ml) a few days thereafter.

When the desired level of differentiation is obtained, the cells can be seeded into the microwells of the devices described herein. The cells in the longitudinal recesses grow, self-assemble, and form three-dimensional tissues. For example, cells in longitudinal recesses and align along the groove portion. The micro-tissues formed in the wells exhibit contractility with greater synchronicity and directionality than two-dimensional monolayers of the same cell type and composition.

The micro-tissues can be evaluated to ascertain or confirm the functional and structural properties of the micro-tissues. For example, after culturing the seeded cells within the microwells, the cells and/or tissues within the microwells can be evaluated to determine whether cells are aligned in the groove portion of one or more of the longitudinal recesses, to determine whether cells have formed three-dimensional structures in one or more longitudinal recesses, or a combination thereof. The cells and/or micro-tissues can also be evaluated to determine whether they are contracting along the longitudinal axis of one or more of the longitudinal recesses. The methods described herein can also include determining micro-tissue morphology, genetic expression, contraction rate, contraction intensity, electrical activity, calcium transient amplitude, intracellular Ca²⁺ level, cell size, contractile force production, sarcomeric a-actinin distribution, or a combination thereof. The micro-tissues can be subjected to a variety of analytical procedures with or without removal of the micro-tissues from the longitudinal recesses.

The efficiency of assays using the methods and devices of this disclosure and suitability for HTS may be evaluated by Z′-factor calculated according to Equation 1.

$${Z}^{′} = 1 - \frac{( 3 {σ}_{p} + 3 {σ}_{n} )}{\vert {μ}_{p} - {μ}_{n} \vert} \qquad Eqn . 1$$

Z′-factor is a dimensionless parameter calculated from measurements of control data and is useful for quantitative assay optimization. It is superior to standard signal-to-noise (S/N) and signal-to-baseline (S/B) metrics, since it takes into account the assay dynamic range (difference in means of positive (μₚ) and negative controls (μₙ)), and standard deviation associated with both positive (σₚ) and negative (σₙ) control data. Techniques for evaluating Z′-factor are described in Zhang, J. H., Chung, T. D. & Oldenburg, K. R. “A Simple Statistical Parameter for Use in Evaluation and Validation of High Throughput Screening Assays.” J Biomol Screen 4, 67-73 (1999). Generally, a Z′-factor greater than 0.5 is preferred, although a lower Z′-factor is not prohibited.

Suitability for HTS techniques may also be assessed by the Z-factor, which assesses the quality of a HTS assay incorporating the influence of the instrumentation (i.e., automation), compound library, and concentrations tested. To calculate Z-factor, the mean (μₛ) and standard deviation (σₛ) of the samples replace the positive controls (μₚ; σₚ) in Equation 1. Thus, the Z-factor may be utilized instead of the Z′-factor for assay optimization when the compound library and automation are used to assess sample variance. Generally, a Z-factor greater than 0.5 is preferred, although a lower Z-factor is not prohibited.

Lactate dehydrogenase (LDH) and cardiac injury biomarkers such as troponin T (TnT) may be used to assess cytotoxicity (membrane rupture) in medium collected from the microwells. LDH will be measured with a colorimetric enzymatic assay (Promega), and TnT via mass spectrometry.

### EXAMPLES

### Example 1: Creating the Heart Micromuscle in COC Microwells

Cells were loaded into the COC wells as a single cell suspension filling each ‘dog bone’ well feature. Within the first few days, cells spread out and attached to the ‘dog bone’ well bottom, pillar features, and side walls. After approximately one week in culture, the cells condensed to form a beating microtissue matching the shape of the ‘dog bone’ COC cast well. Over the next 1-2 weeks the microtissue continued to remodel to form a heart micromuscle fiber spanning between the two sets of pillar arrays. Contractile forces were strong enough to bend pillars with diameters from 30 μm and 70 μm.

COC ‘dog bone’ wells were fit into a standard 96 well plate with four “dog-bone” features per well.

The COC was cleaned/sterilized with 70% ETOH.

A total of 210,000 hiPSC-derived cardiomyocytes (FujiFilm Cellular Dynamics, Inc.) in a 10 μL droplet of plating media was applied to the four dog bone features in each well and allowed to settle by gravity for 10-15 minutes.

Cells distributed evenly within and between the dog bone features. Sedimentation was sufficient to locate most cells within the ‘dog bone’ well bottom.

Excess cells were aspirated, and media was carefully added to wash off cells not settled into the ‘dog bone’ well bottom.

Within the first 4 days the suspension transformed into multiple layers of more elongated cells attaching to the trough bottom, pillars, and side walls.

Within 7 days cells started to form a connected microtissue inside each ‘dog bone’ well, and beating motion was observed. Beating persisted and increased in contractility over the entire cultivation period of 3 weeks.

The tissues initially filled the entire trough area, but over extended culture periods (about day 10 to day 21) the tissues condensed, peeling away from the trough bottom and side walls, forming a heart micromuscle.

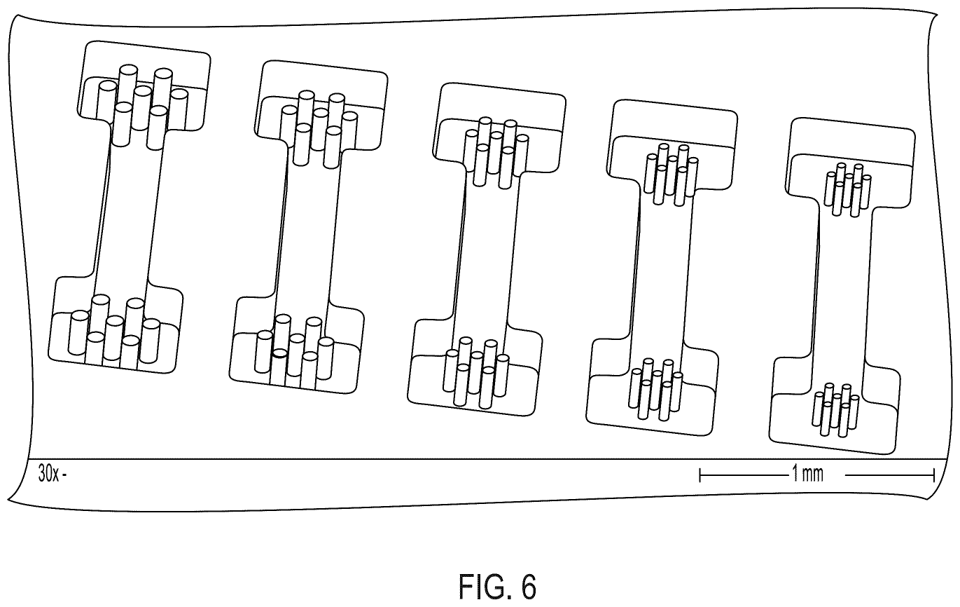

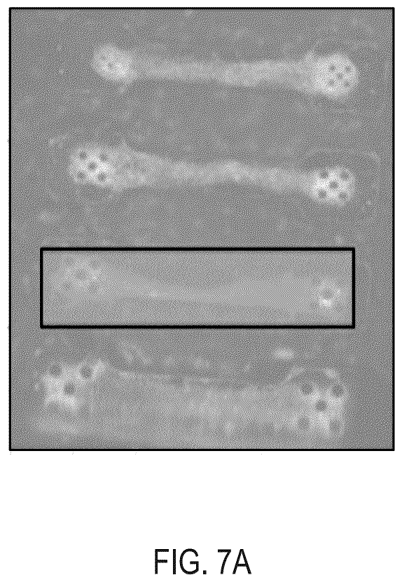

Regardless of pillar diameter or arrangement, the pillar arrays served as anchors and successfully kept the tissues from collapsing towards the center of each trough as shown by fluorescent dye analysis in FIG. 7A.

FIG. 7B shows the contraction of the heart micromuscle a detected by measuring changes in voltage, calcium and motion over time. Each tissue contracted as an independent unit with sufficient force to bend the anchor pillars and be detected by optical microscopy. The magnitude of bending depended on the pillar array layout and pillar diameter.

### Example 2: Fast Acquisition Force of Contraction Across Multiple Tissues

To measure the force of contraction of micro-tissue, the micropillars enable an in situ time resolved characterization of the beating contraction stress. Optical recordings are used for quantitative contraction and electrophysiology measurements. For force analysis, videos of the moving pillars at the focal plane of each pillar tip are acquired. The force of contraction is calculated by first optically capturing the deflection of the micropillars, by using ImageXpress® Micro Confocal system, and then applying beam theory.

High frame rate fluorescence video recordings were taken using reporter fluorescent probes for transmembrane potential (APD) and intracellular calcium handling. Instead of imaging each tissue separately (vertically, one at a time), the camera was configured to simultaneously image four “dog-bone” wells in one image frame for rapid acquisition. Specifically, as shown by the superimposed rectangle in FIG. 8, recordings were taken at a long (2-3 mm) but narrow (below 500 μm) field of view across multiple tissues within one well. As shown in FIG. 8, for force analysis, the field of view encompasses the pillars of each tissue well. The narrow width ensures that fast framerates can be used to acquire to rapid pillar movement. In cases where the imaged area is more important than framerate the field of view can be expanded to full width. Video recordings are cropped in a post processing step to separate signals from different tissues.

### Example 3: Stresses Acting on Pillars in Tested Cardiac Microtissues

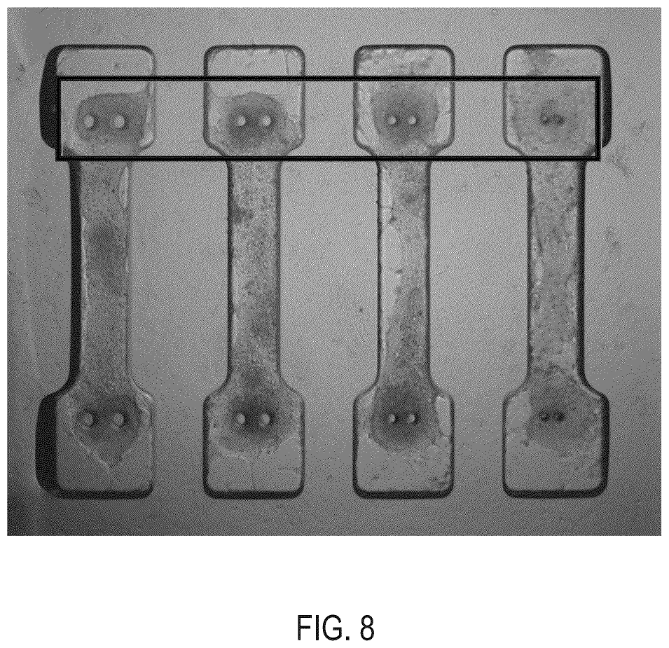

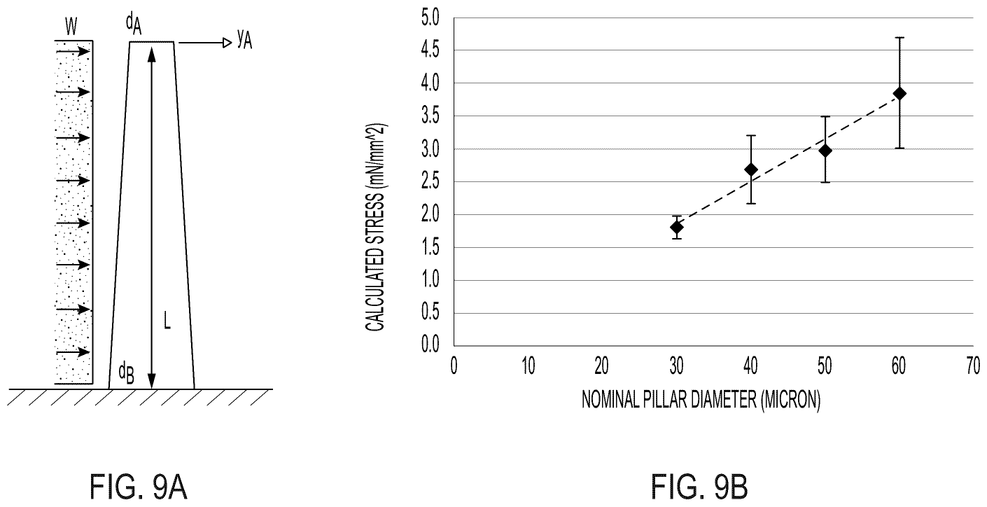

This Example evaluates in vitro modeling of external mechanical load to cardiac tissues. Cardiac microtissues were mounted in a micropillar arranged within the longitudinal recess of a microwell with pillars schematically shown in FIG. 9A.

The force exerted on the pillar area is calculated using Equation 2, which relates the tip displacement to a uniform load on a tapered beam.

$$W = \frac{8 {EI}_{A} {y}_{A}}{{L}^{4}} \frac{4}{{( r - 1 )}^{3}} [ \frac{ln r}{( r - 1 )} - \frac{( 11 {r}^{2} - 7 r + 2 )}{6 {r}^{3}} ] \qquad Equation ( 2 )$$

Where W is the linear force along the length of the pillar, L. E is the modulus of the pillar material, y_{A} is the tip deflection, r is the ratio of the base diameter d_{B} to the tip diameter d_{A}, and I_{A} is the moment of inertia at the pillar tip:

$${I}_{A} = \frac{π}{64} {d}_{A}^{4} \qquad Equation ( 3 )$$

The total calculated force divided by the pillar surface area in contact with the tissue provides a measure of the stress the tissue exerts on the pillars. This calculated stress as a function of nominal pillar diameter is plotted in FIG. 9A, where nominal diameter is the average of the tip and base pillar diameter.

Modeling of external mechanical load to cardiac tissues can be performed either by passive stretch of cardiac tissues mimicking the increase of preload, or stiffening the flexible cantilevers to cardiac tissues mimicking the increase of afterload. Cardiac preload is defined as end-diastolic myocardial wall tension. Here, preload refers to the passive tension exerted by the pillars to the diastolic heart micromuscle at the resting state. The load opposing shortening of the heart micromuscle during contraction is termed cardiac afterload. The cardiac tissue afterload is defined as the pillar tension against the systolic heart micromuscle at the maximal contraction (FIG. 9B right). The afterload or contraction stress increases when the heart micromuscle contracts against the stiffer larger diameter pillars (FIG. 9B right). The maximal range of stress (afterload) is similar to that observed in adult human heart tissue slices (Brandenburger et al. Cardiovasc. Res. 93, 50-59 (2012)), validating the ability of the heart micromuscle to recapitulate the contractile function of the human heart.

Confirmation that pillar diameter affects afterload or contraction stress can be used as a proxy for progressive heart failure. Device designs that include in one well of a 384 plate four ‘dog bone’ wells, each with different pillar diameters, would allow testing a single drug in wells representing different states of heart failure. Heart failure treatments can be tested efficiently to determine the level of heart failure where they are most efficacious.

1. A microwell array configured for high-throughput screening of micro-tissue comprising:
a substrate;
a plurality of wells in the substrate, each well having an opening, a sidewall surface and a bottom surface;
at least one longitudinal recess arranged in the bottom surface of each of the plurality of wells; and
at least one micropillar arranged within the longitudinal recess at each end of the longitudinal recess.

2. The microwell array of claim 1, further comprising an expanded depression arranged at each end of the at least one longitudinal recess.

3. The microwell array of claim 2, wherein the expanded depression is circular, oval, rectangular, square, V-shaped, or triangular.

4. The microwell array of claim 1, further comprising a set of electrodes electrically connected to at least one of the plurality of wells.

5. The microwell array of claim 4, wherein the electrodes are composed of evaporated gold.

6. The microwell array of claim 1, wherein the at least one longitudinal recess comprises a groove portion.

7. The microwell array of claim 1, wherein a plurality of micropillars are arranged within the at least one longitudinal recess at each end of the longitudinal recess.

8. The microwell array of claim 7, wherein the plurality of micropillars is arranged in a triagonal pattern.

9. The microwell array of claim 1, wherein an elastic modulus of a material of the at least one micropillar is 30 to 70 MPa.

10. The microwell array of claim 1, wherein at least one well of the microwell array comprises a plurality of discontinuous longitudinal recesses.

11. The microwell array of claim 1, wherein a ratio of a length to a width of the at least one longitudinal recess is 2 to 15.

12. The microwell array of claim 1, wherein the micropillars are confined to the ends of the longitudinal recess.

13. The microwell array of claim 1, wherein a diameter of the at least one micropillar is 70 μm or less.

14. The microwell array of claim 1, wherein a depth of at least one longitudinal recess is 500 μm or less.

15. The microwell array of claim 1, wherein a distance between the micropillars at each end of the longitudinal recess is 1000 to 2000 μm.

16. The microwell array of claim 6, wherein the width of the groove portion of at least one longitudinal recess is less than 200 μm.

17. The microwell array of claim 2, wherein a width of the expanded depression is more than 200 μm and less than 600 μm.

18. The microwell array of claim 1, wherein at least a portion of a bottom surface of at least one longitudinal recess is provided with a non-fouling coating.

19. The microwell array of claim 1, wherein a cell adhesion coating is provided on the bottom surface at least at each end of the at least one longitudinal recess in an area adjacent the at least one micropillar.

20. The microwell array of claim 1, wherein the substrate is composed of cyclic olefin copolymer.

21. The microwell array of claim 1, wherein a portion of the bottom surface of the well connecting to a recess side wall of the longitudinal recess is provided with a sloped portion having an angle of incline such that a depth of the bottom surface of the well in the sloped portion increases with closer proximity to the longitudinal recess.

22. A method of preparing high-throughput screening mammalian micro-tissue model comprising:
a) providing a microwell array having:
a substrate;
a plurality of wells in the substrate, each well having an opening, a sidewall surface and a bottom surface;
at least one longitudinal recess arranged in the bottom surface of each of the plurality of wells; and
at least one micropillar arranged within the longitudinal recess at each end of the longitudinal recess;
b) seeding mammalian cells into the at least one longitudinal recess;
c) culturing the seeded cells within the at least one longitudinal recess to thereby induce self-assembly of the mammalian cells into one or more uniaxially aligned micro-tissues,
wherein each end of the micro-tissue is secured in the at least one longitudinal recess by the at least one micropillar at each end of the longitudinal recess.

23. The method of claim 22, wherein about 2000 to about 9500 cells are seeded into each of the plurality of wells.

24. The method of claim 22, wherein a depth of the at least one longitudinal recess is 500 μm or less.

25. The method of claim 22, wherein a plurality of micropillars are arranged within the at least one longitudinal recess at each end of the longitudinal recess.

26. The method of claim 22, wherein at least one well of the microwell array comprises a plurality of discontinuous longitudinal recesses in the bottom surface of the at least one well.

27. The method of claim 22, wherein the seeded cells are a mixture of mammalian cell types typically present in a mammalian organ.

28. The method of claim 27, wherein the mammalian organ is selected from the group consisting of heart, muscle, and neuronal tissue.

29. The method of claim 22, wherein seeding the mammalian cells comprises settling the cells into the longitudinal recesses by gravity.

30. The method of claim 22, further comprising introducing a test compound, oligonucleotide, nucleic acid, protein, lipid nanoparticle, or a combination thereof into one or more wells while culturing the seeded cells within the longitudinal recesses.

31. The method of claim 22, further comprising imaging the microwell array wherein a field of view includes the end of each of a plurality of longitudinal recess within one well.

32. The method of claim 31, wherein the field of view captures only one of the two ends of the plurality of longitudinal recesses.

33. The microwell array of claim 1, wherein at least a bottom surface and sidewall of the at least one longitudinal recess is provided with a non-fouling coating.
